(19) United States
(12) Patent Application Publication
(10) Pub. No.: US 2009/0234125 A1
(43) Pub. Date: Sep. 17, 2009

(54) Amorphous retapamulin and processes for preparation thereof

(76) Inventors: Eli Lancry, Modiin (IL); Lilach Hedvati, Doar Na Hefer (IL); Greta Sterimbaum, Rishon-Lezion (IL); Ariel Mittelman, Elad (IL); Tali Katav, Sharon (IL)

Correspondence Address: KENYON & KENYON LLP, ONE BROADWAY, NEW YORK, NY, 10004, US

(21) Appl. No.: 12/313,962

(22) Filed: Nov. 26, 2008

Related U.S. Application Data
(60) Provisional application No. 61/004,384, filed on Nov. 26, 2007.
(60) Provisional application No. 61/123,519, filed on Apr. 8, 2008.
(60) Provisional application No. 61/126,297, filed on May 1, 2008.
(60) Provisional application No. 61/188,186, filed on Aug. 6, 2008.

(51) Int. Cl. C07D 451/06

(52) U.S. Cl. 546/128

(57) ABSTRACT

An amorphous form of Retapamulin, preferably in powder form, and processes for preparation thereof, are provided. Amorphous Retapamulin of the present invention can contain less than about 10 percent crystallinity, preferably less than about 5 percent crystallinity. Pharmaceutical compositions comprising amorphous Retapamulin are also provided.

### CROSS REFERENCE TO RELATED APPLICATIONS

This application claims the benefit of U.S. Provisional Application Nos. 61/004,384 filed Nov. 26, 2007; 61/123,519 filed Apr. 8, 2008; 61/126,297 filed May 1, 2008 and 61/188,186 filed Aug. 6, 2008; hereby incorporated by reference.

### FIELD OF THE INVENTION

The present invention relates to solid state chemistry of Retapamulin.

### BACKGROUND OF THE INVENTION

5-Acetic acid, [[(3-exo)-8-methyl-8-azabicyclo[3.2.1]oct-3-yl]thio]-(3aS,4R,5S,6S,8R,9R,9aR,10R)-6-ethenyldecahydro-5-hydroxy-4,6,9,10-tetramethyl-1-oxo-3a,9-propano-3aH-cyclopentacycloocten-8-yl ester, whose international nonproprietary name is Retapamulin [CAS number: 224452-66-8], has the following chemical structure:

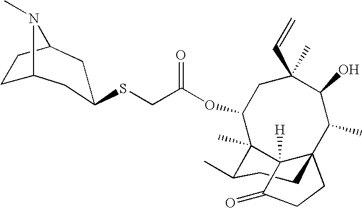

Retapamulin, first disclosed in U.S. Pat. No. 6,281,226, is used in the treatment of secondarily-infected traumatic lesions (SITL). Processes for preparing pleuromutilin derivatives have been disclosed in U.S. Patent No. 2006/0276503. The present invention relates to the solid state physical properties of Retapamulin. These properties can be influenced by controlling the conditions under which Retapamulin is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a formulation, which may necessitate the use of glidants such as colloidal silicon dioxide, talc, starch or tribasic calcium phosphate.

Another important solid state property of a pharmaceutical compound is its rate of dissolution in aqueous fluid, syrups, elixirs, ointment and other liquid medicaments. The solid state form of a compound may also affect its behavior on compaction and its storage stability.

Crystalline Retapamulin has been described in U.S. Patent Application Publication No. US2006/0276503 and in International Patent Application Publication WO 2005/023257, while a second polymorph of Retapamulin have been referred to in WO 2006/092334.

The discovery of new solid states of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristics.

### SUMMARY OF THE INVENTION

The present invention encompasses an amorphous form of Retapamulin, preferably in powder form, and processes for preparation thereof.

Amorphous Retapamulin of the present invention can contain less than about 10 percent crystallinity, preferably less than about 5 percent crystallinity.

In one specific embodiment, amorphous Retapamulin of the present invention comprises less than about 5 percent of crystalline Retapamulin wherein the crystalline form is characterized by a PXRD pattern with peaks at about 9.6, about 12.8, about 13.9 and about 19.6±0.2 degrees 2θ. Preferably, the amorphous Retapamulin of the present invention comprises less than about 3 percent of the above crystalline Retapamulin, and more preferably, less than about 1 percent of said crystalline Retapamulin, as percent area XRD.

The present invention further encompasses a process for preparing a pharmaceutical formulation comprising combining amorphous Retapamulin of the present invention with at least one pharmaceutically acceptable excipient.

The present invention further encompasses the use of amorphous Retapamulin of the present invention for the manufacture of a pharmaceutical composition.

The present invention further encompasses the use of amorphous Retapamulin made by the processes of the invention for the manufacture of a pharmaceutical composition.

### BRIEF DESCRIPTION OF THE FIGURES

FIG. 1 represents a powder X-ray diffraction pattern for amorphous Retapamulin.

FIG. 2 represents a photomicrograph of amorphous Retapamulin.

FIG. 3 represents a photomicrograph of crystalline Retapamulin.

### DETAILED DESCRIPTION OF THE INVENTION

As used herein, the terms “powder” or “powdery” refer to a solid compound in the form of particles or granules where the particles or granules can be poured. Preferably, the powders are solid, loose, dry particles.

As used herein, the term “crystalline Retapamulin” refers to a crystalline form of Retapamulin characterized by a PXRD pattern with peaks at about 9.6, about 12.8, about 13.9 and about 19.6±0.2 degrees 2θ, as presented in US 2006/0276503.

As used herein, the term “vacuum” refers to a reduced pressure of below about 100 mm Hg, more preferably, below about 50 mm Hg, and, most preferably, below about 30 mm Hg.

As used herein, the term “reduced pressure” refers to a pressure below 760 mm Hg or 1 atmosphere.

As used herein, the term “room temperature” refers to a temperature of about 20° C. to about 35° C., more preferably about 20° C. to about 25° C. and most preferably about 25° C.

As used herein, the term “therapeutically effective amount” means the amount of the amorphous Retapamulin of the present invention that, when administered to a patient for treating a disease or other undesirable medical condition, is sufficient to have a beneficial effect with respect to that disease or condition. The “therapeutically effective amount” will vary depending on the disease or condition and its severity, and the age, weight, etc., of the patient to be treated. Determining the therapeutically effective amount is within the ordinary skill of the art, and requires no more than routine experimentation.

The present invention encompasses an amorphous form of Retapamulin and processes for preparation thereof. We have also found that amorphous Retapamulin can be obtained in the form of a powder, which is desirable for formulation.

In one embodiment of the present invention, amorphous Retapamulin is presented. The amorphous form may comprise less than about 10 percent crystallinity, preferably less than about 5 percent crystallinity.

The percent of crystallinity can be determined by dividing the total area of the peaks arising from the crystalline fraction of the sample with the total area of the sample's diffractogram.

In another embodiment of the present invention, amorphous Retapamulin is presented comprising less than about 5 weight percent of crystalline Retapamulin, preferably less than about 3 weight percent of crystalline Retapamulin, and more preferably, less than about 1 weight percent of crystalline Retapamulin.

In another embodiment, the present invention encompasses a process for preparing amorphous Retapamulin comprising: providing a solution of Retapamulin in a solvent selected from C₁ to C₄ alcohols or dichloromethane; and removal of solvent to obtain amorphous Retapamulin. The solution of Retapamulin can be prepared by dissolving in the selected solvent. The dissolution of Retapamulin in the solvent can be carried out at room temperature or dissolution can be assisted by heating to a temperature of about 30° C. to about reflux, preferably about 40° C. to about 60° C. Preferably, the C₁ to C₄ alcohols are methanol, ethanol or a mixture thereof. The ratio of Retapamulin to solvent can be in a ratio of about 1:1 to about 1:20, preferably about 1:8 to about 1:15 (grams/ml).

Solvent removal may be by a number of means such as evaporation, including fast evaporation (see e.g. US2005/0272768, incorporated herein by reference) and spray drying. Solvent removal is usually complete after dryness. Preferably, solvent removal is performed under vacuum.

Spray-drying broadly refers to processes involving breaking up liquid mixtures into small droplets, preferably by atomization, and rapidly removing solvent from the mixture. In a typical spray-drying apparatus, there is a strong driving force for evaporation of solvent from the droplets, which may be provided by a heated drying gas. Spray-drying processes and equipment are described in Perry's CHEMICAL ENGINEER'S HANDBOOK, pgs. 20-54 to 20-57 (Sixth Edition 1984).

By way of non-limiting example only, the typical spray-drying apparatus comprises a drying chamber, an atomizer for atomizing a solvent containing feed into the drying chamber, a source of heated drying gas that flows into the drying chamber to remove solvent from the atomized solvent containing feed, an outlet for the products of drying, and a product collector, located downstream of the drying chamber. Examples of such apparatuses include Niro Models PSD-1, PSD-2, and PSD-4 (Niro A/S, Soeborg, Denmark). Typically, the product collector includes a cyclone connected to the drying apparatus. In the cyclone, the particles produced during spray-drying are separated from the drying gas and evaporated solvent, allowing the particles to be collected. A filter may also be used to separate and collect the particles produced by spray-drying. The process of the invention is not limited to the use of such drying apparatuses as described above.

The gas inlet temperature during spray drying is about 35° C. to about 70° C. More preferably, the gas inlet temperature is about 40° C. to about 67° C. An “inlet temperature” is the temperature at which the solution enters the spray dryer.

The outlet temperature is preferably below the inlet temperature, more preferably, the outlet temperature is from about 20° C. to about 45° C. Most preferably, the outlet product is from about 25° C. to about 42° C. An “outlet temperature” is the temperature at which the gas exits the spray dryer.

Inlet or outlet temperatures may be varied, if necessary, depending on the equipment, gas, or other experimental parameters. For example, it is known that the outlet temperature may depend on parameters such as aspirator rate, air humidity, inlet temperature, spray air flow, feed rate or concentration.

In one embodiment, the present invention encompasses a process for preparing amorphous Retapamulin by a fast evaporation process comprising dissolving Retapamulin in an organic solvent, feeding the solution into a chamber maintained at a reduced pressure (pressure of less than one atmosphere) and a temperature of less than about 100° C. until obtaining a precipitate. The temperature can be about 50° C. to about 100° C. Preferably, the solvent is selected from the group consisting of: C₁ to C₄ alcohols, C₃ to C₇ ketones, C₃ to C₇ esters, C₅ to C₇ straight or cyclic saturated hydrocarbons or C₄ to C₈ ethers, C₂ to C₆ nitriles and mixtures thereof. More preferably, the solvent is selected from the group consisting of: methanol, ethanol, acetone, toluene, acetonitrile, ethyl acetate, heptane, hexane, diethyl ether, methyl isobutyl ether, di-isopropyl-ether and mixtures thereof. Most preferably, the solvent is selected from the group consisting of: methanol, ethanol and dichloromethane.

The amount of crystallinity may be quantified by methods known in the art like “crystallinity index” available to most XRD softwares.

Generally, the detection of peaks of crystalline Retapamulin in amorphous Retapamulin can be done by any method known to the skilled artisan.

For example, a person skilled in the art would know, when using XRD as a method for detecting or quantifying peaks of crystalline Retapamulin in amorphous Retapamulin, to select a peak or a number of peaks from the following list of peaks: about 9.6, 12.8, 13.9 and 19.6±0.2 degrees 2θ. The absence or presence or intensity of a peak or a number of peaks from the following list of peaks: about 9.6, 12.8, 13.9 and 19.6±0.2 degrees 2θ, may be monitored at a scan rate slow enough, according to the common knowledge of those skilled in the art. The scan rate used may vary from instrument to instrument, and sample preparation. A skilled artisan will know to use other accepted analytical methods such as solid-state NMR, Raman, or IR to detect crystalline Retapamulin in amorphous Retapamulin.

The present invention further encompasses a solid containing pharmaceutical composition comprising amorphous Retapamulin of the present invention and at least one pharmaceutically acceptable excipient. In some embodiments, the pharmaceutical composition is completely solid.

The present invention further encompasses a process for preparing a solid containing pharmaceutical formulation comprising combining amorphous Retapamulin of the present invention with at least one pharmaceutically acceptable excipient. In some embodiments, the pharmaceutical formulation is completely solid.

The present invention further encompasses the use of amorphous Retapamulin of the present invention for the manufacture of a solid containing pharmaceutical composition.

The present invention further encompasses the use of amorphous Retapamulin made by the processes of the invention, for the manufacture of a solid containing pharmaceutical composition.

Methods of administration of a pharmaceutical composition of the present invention may comprise administration in various preparations depending on the age, sex, and symptoms of the patient.

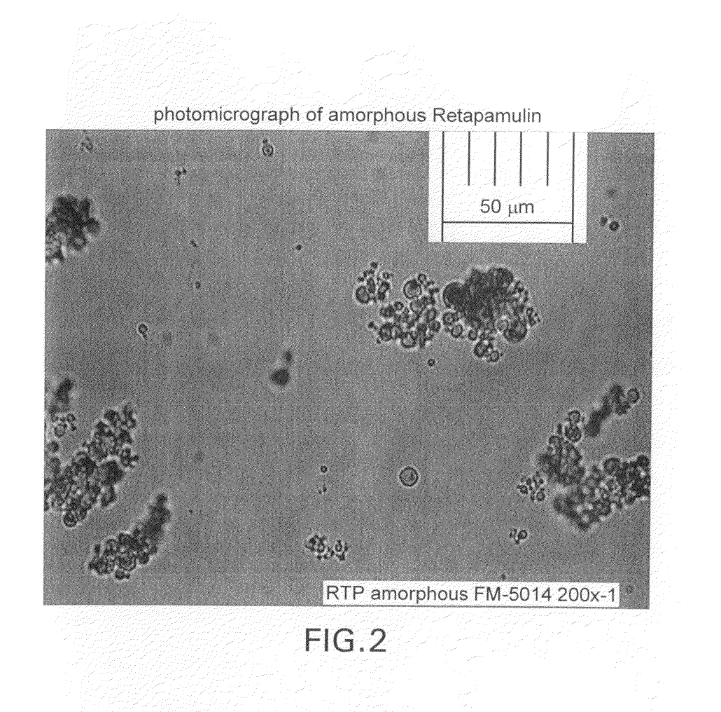

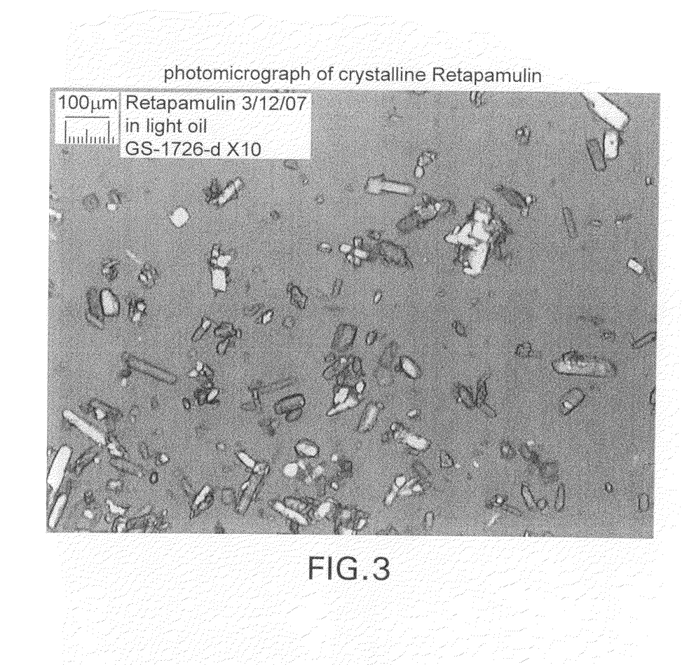

Amorphous Retapamulin has spherical particles, with less than 20 μm diameter, while crystalline Retapamulin forms rod-shaped crystals, with length in the order of 100 μm. See FIGS. 2 and 3.

The bulk properties of the amorphous form of Retapamulin are advantageous compared to those of the prior art, crystalline Retapamulin. The flowability of materials with spherical particles is better than the flowability of those with rod shaped particles. Flowability is a very important factor for the manufacturing process, as it affects all the processes that involve powder-handling, including blending, feeding, compaction and fluidization. The lower particle size of amorphous Retapamulin is also advantageous in comparison to crystalline Retapamulin, in particular for preparing homogenous ointment.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Instruments

### XRD

Powder X-ray diffraction (“XRD”) analysis can be carried out using any XRD powder diffractometer commonly used in the industry. The Retapamulin samples of this invention were run in a SCINTAG powder X-ray diffractometer model X'TRA equipped with a solid-state detector. Copper radiation of λ=1.5418 Angstroms. The sample can be introduced using a round standard aluminum sample holder with round zero background quartz plate in the bottom and is scanned by a continuous scan at a rate of 3° per minute

### A. Preparation of Amorphous Retapamulin

### Example 1

A flask was loaded with 10 ml methanol, and Retapamulin (1 g). The mixture was heated to 45° C. and stirred until dissolution. The solvent was evaporated to dryness. Amorphous Retapamulin was obtained (powdery material).

### Example 2

A flask was loaded with 10 ml CH₂Cl₂, and 1 g Retapamulin. The mixture was heated to 45° C. and stirred until dissolution. The solvent was evaporated to dryness. Amorphous Retapamulin was obtained (powdery material).

### Example 3

Retapamulin (10 g) was dissolved in methanol (100 ml), the solution was pumped into a spray dryer at room temperature, nitrogen was used as the drying gas at an inlet temperature of 40° C. The evaporated solvent, product and nitrogen exited the spray dryer at 25-30° C. Amorphous Retapamulin was obtained (powdery_material).

### Example 4

Retapamulin (15 g) was dissolved in methanol (150 ml), the solution was pumped into a spray dryer at room temperature, nitrogen was used as the drying gas at an inlet temperature 57-59° C. The evaporated solvent, product and nitrogen exited the spray dryer at 37-42° C. Amorphous Retapamulin was obtained (powdery_material).

### Example 5

Retapamulin (5 g) was dissolved in ethanol (50 ml), the solution was pumped into a spray dryer at room temperature, nitrogen was used as the drying gas at an inlet temperature 65-67° C. The evaporated solvent, product and nitrogen exited the spray dryer at 39-42° C. Amorphous Retapamulin was obtained (powdery_material).

### Example 6

Retapamulin (6 g) was dissolved in ethanol (30 ml) and methanol (30 ml), the solution was pumped into a spray dryer at room temperature, nitrogen was used as the drying gas at an inlet temperature 55-58° C. The evaporated solvent, product and nitrogen exited the spray dryer at 37-39° C. Amorphous Retapamulin was obtained (powdery_material).

### Example 7

Retapamulin (2 g) is dissolved in methanol (2-50 vol) the solution is evaporated to a volume of 2 ml. The solution is injected through a syringe needle into a flask under vacuum and heated to 50° C. The solid amorphous Retapamulin is solidified and collected in the bottom of the flask.

### B. Preparation of a Formulation of Amorphous Retapamulin

0.0033 g of amorphous Retapamulin was dispersed uniformly in 3.066 g petrolatum.

1. Amorphous Retapamulin.

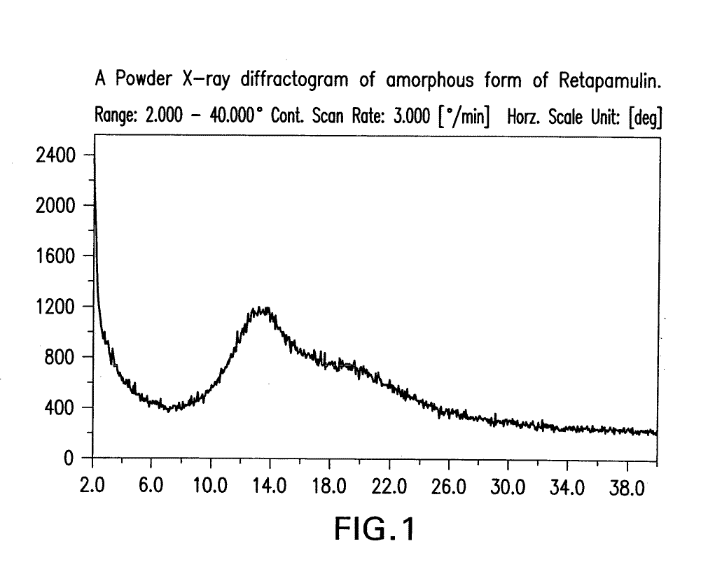

2. The amorphous Retapamulin of claim 1, characterized by an X-ray powder diffraction pattern as presented in FIG. 1.

3. The amorphous Retapamulin of claim 1, wherein the amorphous form is in powder form.

4. The amorphous Retapamulin of claim 1, wherein the amorphous form has less than about 5 weight percent of a crystalline form of Retapamulin, wherein the crystalline form is characterized by a PXRD pattern with peaks at about 9.6, about 12.8, about 13.9 and about 19.6.

5. The amorphous Retapamulin of claim 4, wherein the amorphous form has less than about 3 weight percent of the crystalline form of Retapamulin.

6. The amorphous Retapamulin of claim 5, wherein the amorphous Retapamulin has less than about 1 weight percent of crystalline Retapamulin.

7. A process for preparing the amorphous Retapamulin of claim 1, comprising providing a solution of Retapamulin in a solvent selected from C₁ to C₄ alcohols or dichloromethane; and removing the solvent to obtain amorphous Retapamulin.

8. The process of claim 7, wherein the solvent is a C₁ to C₄ alcohol.

9. The process of claim 7, wherein the solvent is methanol, ethanol or a mixture thereof.

10. The process of claim 7, wherein the solvent is dichloromethane.

11. The process of claim 7, wherein the ratio of Retapamulin to solvent is in a ratio of about 1:1 to about 1:20 (grams/ml).

12. The process of claim 7, wherein the ratio of Retapamulin to solvent is in a ratio of about 1:8 to about 1:15 (grams/ml).

13. The process of claim 7, wherein removing is carried out by evaporation.

14. The process of claim 7, wherein removing is carried out by spray drying.

15. The process of claim 14, wherein spray drying is carried out with an inlet temperature of about 35° C. to about 70° C.

16. A process for preparing amorphous Retapamulin of claim 1 comprising dissolving Retapamulin in an organic solvent, feeding the solution into a chamber maintained at a reduced pressure and a temperature of less than about 100° C. to obtain a precipitate.

17. The process of claim 16, wherein the temperature is about 50° C. to about 100° C.

18. The process of claim 16, wherein the solvent is selected from the group consisting of: C₁ to C₄ alcohols, C₃ to C₇ ketones, C₃ to C₇ esters, C₅ to C₇ straight or cyclic saturated hydrocarbons or C₄ to C₈ ethers, C₂ to C₆ nitriles and mixtures thereof.

19. The process of claim 16, wherein the solvent is selected from the group consisting of: methanol, ethanol, acetone, toluene, acetonitrile, ethyl acetate, heptane, hexane, diethyl ether, methyl isobutyl ether, di-isopropyl-ether and mixtures thereof.

20. The process of claim 16, wherein the solvent is selected from the group consisting of: methanol, ethanol and dichloromethane.

21. A solid containing pharmaceutical formulation comprising the amorphous Retapamulin according to any of claims 1-6.

22. Amorphous Retapamulin according to any of claims 1-6 for use as a medicament.

23. Amorphous Retapamulin according to claim 22 for use as a medicament for the treatment of secondarily-infected traumatic lesions.

24. Use of amorphous Retapamulin according to any of claims 1-6 for the manufacture of a medicament for treatment of secondarily-infected traumatic lesions.
